(19) United States
(12) Patent Application Publication
(10) Pub. No.: US 2011/0276132 A1
(43) Pub. Date: Nov. 10, 2011

(54) METHOD AND IMPLANT FOR REFRACTIVE ERROR CORRECTION

(76) Inventor: Bartlomiej Kaluzny, Bydgoszcz (PL)

(21) Appl. No.: 13/097,512

(22) Filed: Apr. 29, 2011

(30) Foreign Application Priority Data
May 4, 2010 (PL) P391143

(51) Int. Cl. A61F 2/14

(52) U.S. Cl. 623/5.16

(57) ABSTRACT

The patentable subject matter is the method and implant used in refractive error correction, in particular in short-sightedness, hypermetropia, astigmatism and presbyopia treatment consisting in a new method and implant causing changes to the curvature of the front surface of the cornea through a controlled increase in the corneal epithelium thickness, the method being implemented using a special implant in the form of an intraepithelial scaffold having a porous structure.

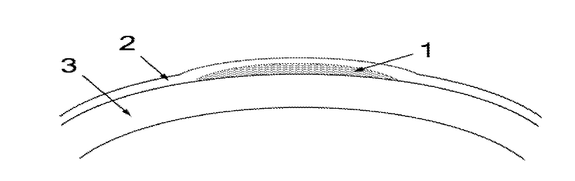

The patentable subject matter is the method and implant used for refractive error correction, particularly in short-sightedness, hypermetropia, astigmatism and presbyopia treatment.

Literature describes a number of refractive error correction methods of which the most common are glasses and contact lenses. In the recent years, surgical correction of eye refraction—refractive surgery—has gained in importance. Vast majority of procedures consisting in removing of part of the corneal tissue are based on the use of excimer or femtosecond laser to change the front curvature of the cornea with the aim to correct an existing refractive error.

The main drawback of laser-based methods is the fact that a substantial amount of tissue from deeper layers of the cornea—stroma—is removed. This results in the weakening of the cornea's biomechanics leading, in some cases, to serious uncontrolled disturbances in its shape. Another major shortcoming of laser-based correction methods is that such procedures are irreversible as the corneal stroma does not regenerate. It poses a serious problem in the event a procedure has been performed in an incorrect way or the patient is not satisfied with it for some reasons.

There are also methods for correcting refractive errors involving artificial lenses as well as rings and discs with stenopeic foramens which are implanted in the corneal stroma allowing short-sightedness or presbyopia to be corrected respectively. These procedures, however, involve the cutting of the cornea, which makes them only partially reversible as well as entailing the use of expensive femtosecond laser.

The cornea is the front transparent part of the eyeball wall characterised by slightly larger curvature. The thinnest part of the cornea is its centre, where its average thickness ranges from 500 to 600 μm. The most peripheral layer is the stratified non-cornifying squamous epithelium which consists in the centre of 5-6 layers and is around 50-60 μm thick. New cells are created by meiotic division of stem t cells located in Vogt palisades located in the corneal limbus. Underneath the epithelium is the Bowman's layer—acellular and composed of collagen fibres—with a thickness of 8-12 μm, stroma or proper substance, making up 90% of the cornea thickness and the Descemet's membrane located at the outer boundary of the stroma constituting the basement layer for endothelium cells. The only layer of the cornea capable of regenerating is the epithelium. Following eye injury, insignificant epithelial loss heals within several hours and the total cornea epithelium regeneration cycle lasts 7 days. Clinical studies using optical tomography, among others, have shown that in the areas where the Bowman's layer and stroma become excavated for various reasons, the epithelium can be many times thicker. The above shows that the corneal epithelium may be considerably thicker in certain areas without losing its function. Appropriate conditions must be met to make it possible.

The essence of the invention consists in developing a new method and implant used for refractive error correction involving changes in the curvature of the front corneal surface through a controlled increase in the thickness of the corneal epithelium, the method being implemented through the use of a special implant in the form of an intraepithelial scaffold having a porous structure.

The advantage of the invention is that it offers the possibility of short-sightedness, hypermetropia, astigmatism and presbyopia correction, the procedure itself being fully reversible, i.e. the intraepithelial implant can be removed together with the surrounding epithelium if necessary and the post-procedural wound will be then covered with the regenerating epithelium within several days, restoring corneal surface to the condition from before the procedure. The said solution can be also used in treating many other corneal diseases affecting in particular the surface and shape of the cornea.

The invention-based method is described in more detail in the appended schematic drawing in the method implementation description.

Drawing presents cross-section of a fragment of the eye cornea, an implant 1, corneal epithelium 2 and corneal stroma 3.

### IMPLEMENTATION OF THE INVENTION-BASED METHOD

The invention-based method is implemented by removing mechanically the corneal epithelium 2. Subsequently, an implant in the form of an intraepithelial scaffold 1 having porous and spongy structure is placed on the uncovered surface of the corneal stroma 3 (Bowman's layer). The implant, after it has penetrated through the regenerating epithelium, causes the epithelial cells which outgrow the scaffold to change the curvature of the anterior corneal surface.

The intraepithelial scaffold is shaped so that it appropriately clings to the Bowman's layer on one side and causes desired changes in the shape of the anterior surface of the cornea on the other side which is a prerequisite for refractive error correction. The intraepithelial scaffold 1 has a spongy structure with the size of the pores exceeding the size of the epithelium basal cells—over 20 μm in diameter—enabling free migration of epithelial cells and nutrients. The implant material has very high biocompatibility and transparency properties also when filled by the epithelial cells of the cornea.

1. The refractive error correction method characterised in that it consists in mechanical removal of the corneal epithelium 2 and, subsequently, placement of an implant 1 on the uncovered surface of the corneal stroma 3 (Bowman's layer) which, after it has penetrated through the regenerating epithelium, causes changes in the curvature of the front surface of the cornea.

2. The refractive error correcting implant characterised in that it has the form of a transparent intraepithelial scaffold having profiled surfaces and porous, spongy structure with pores diameter above 20 μm, implant material being fully biocompatible.
